(19) United States
(12) Patent Application Publication
(10) Pub. No.: US 2023/0106102 A1
(43) Pub. Date: Apr. 6, 2023

(54) METHOD OF PRODUCING TRICHODESMA KHASIANUM CLARKE LEAF EXTRACTS AND APPLICATION OF THE SAME

(71) Applicant: BAI-CHUNG-REN Foodstuff.Co.Ltd, (US)

(72) Inventors: CHE-HUANG HSU, Taitung City (TW); JHEN-YI WANG, Taitung City (TW)

(73) Assignee: BAI-CHUNG-REN Foodstuff.Co.Ltd, Taitung City (TW)

(21) Appl. No.: 17/744,569

(22) Filed: May 13, 2022

(30) Foreign Application Priority Data
Sep. 24, 2021 (TW) 110135614

(51) Int. Cl. A23L 5/20; A61P 3/06

(52) U.S. Cl. CPC A23L 5/23; A61P 3/06

(57) ABSTRACT

A method of producing trichodesma khasianum clarke leaf extracts. The trichodesma khasianum clarke leaf extracts contains: flavonoids and rosmarinic acid. When producing the trichodesma khasianum clarke leaf extracts, a concentration of alcohol is 50% to 60%, an extracting time is 1 hour to 1.5 hours, and an extracting temperature is 60° C. to 75° C., wherein the trichodesma khasianum clarke leaf extracts are configured to regulate a serum liver function index, a serum cholesterol, and a triglyceride in a user's body.

### FIELD OF THE INVENTION

The present invention relates to trichodesma khasianum clarke leaf extracts which are configured to regulate a serum liver function index, a serum cholesterol, and a triglyceride in a user's body.

### BACKGROUND OF THE INVENTION

False physalis is also known as Bicago, the scientific name is Trichodesma khasianum Clarke, the British name is Khasya trichodesma, and it is a plant of the Boraginaceae family. False physalis is produced in low-and middle-altitude plains and mountainous areas in Taiwan. However, those who grow in mountainous areas have larger leaves and dark green. The leaves of this plant are commonly used food materials by the aboriginal people. During festivals, the leaves are used to wrap the steamed rice cake food A Bai, which contains a lot of plant fiber to help digestion, and is a special usage. When eating, it is eaten with the leaves, which has a light fragrance. This method is different from those using moon peach or banana leaves as the substrate, and has a different flavor.

In the past research application of trichodesma khasianum clarke leaf, it have done experiments on the protective effect of acute gastric mucosal injury. In this experiment, it learned that trichodesma khasianum clarke leaf 80% ethanol extracts (80% ethanol extracts of trichodesma khasianum clarke leaf, 80EETC) Has the highest content of total polyphenols and flavonoids, as well as the best antioxidant capacity. HPLC and Q Exactive Focus LC-HRMS analysis showed that the main component of 80EETC is rosmarinic acid (RA). 80EETC inhibits the expression of iNOS and COX-2 in RAW 264.7 macrophages induced by LPS, thereby reducing the pro-inflammatory hormones NO and PGE2. generate. The results of the gastric mucosal protective cell test showed that 80EETC can slow down the ethanol-induced RGM-1 gastric mucosal cell damage by increasing heat shock proteins (HSPs), reducing ROS, and inhibiting pro-apoptotic proteins. It is known from the cell wound healing experiment that 80EETC can promote wound healing, confirming that 80EETC has the potential to resist inflammation, protect the survival of gastric mucosal cells and promote wound healing.

Furthermore, the trichodesma khasianum clarke leaves have not been applied in other biotechnological fields. Therefore, the fake physalis leaves that are often eaten by the aborigines are studied in the field of greater biotechnology, which has expected economic benefits and value.

The present invention has arisen to mitigate and/or obviate the afore-described disadvantages.

### SUMMARY OF THE INVENTION

The primary aspect of the present invention is to provide trichodesma khasianum clarke leaf extracts which are configured to regulate a serum liver function index, a serum cholesterol, and a triglyceride in a user's body.

Another aspect of the present invention is to provide trichodesma khasianum clarke leaf extracts which are configured to 10 regulate a serum liver function index, a serum cholesterol, and a triglyceride in a user's body.

To obtain the above aspects, a method of producing trichodesma khasianum clarke leaf extracts provided by the present invention, the trichodesma khasianum clarke leaf extracts contains: flavonoids and rosmarinic acid.

When producing the trichodesma khasianum clarke leaf extracts, a concentration of alcohol is 50% to 60%, an extracting time is 1 hour to 1.5 hours, and an extracting temperature is 60° C. to 75° C.

Preferably, the trichodesma khasianum clarke leaves are dried and chopped coarsely before extracting trichodesma khasianum clarke leaves, then the trichodesma khasianum clarke leaves are extracted by using alcohol.

Preferably, after extracting the trichodesma khasianum clarke leaves, the trichodesma khasianum clarke leaves are vacuumed, concentrated, frozen, dried, pelletized in a fluid layer, granulated, and tableting pressed to produce the trichodesma khasianum clarke leaf extracts which are edible.

Preferably, sea salt, sugar, and mint are added into the trichodesma khasianum clarke leaf extracts to enhance taste.

### BRIEF DESCRIPTION OF THE DRAWINGS

FIG. 1 is a diagram view showing an effect of trichodesma khasianum clarke leaf extracts on a body weight, a liver weight, and a ratio of the liver weight/the body weight of alcoholic liver in mice according to a preferred embodiment of the present invention.

FIG. 2 is a diagram view showing an effect of trichodesma khasianum clarke leaf extracts on aspartate aminotransferase (AST) and alanine aminotransferase (ALT) activities of alcoholic liver injury index in the mice according to the preferred embodiment of the present invention.

FIG. 3 is a diagram view showing an effect of trichodesma khasianum clarke leaf extracts on a pathological section of an alcoholic liver injury index in the mice according to the preferred embodiment of the present invention.

### DETAILED DESCRIPTION OF THE PREFERRED EMBODIMENTS

In previous studies, it can be understood that trichodesma khasianum clarke leaf have the potential to fight inflammation and

promote wound healing, so further studies on the ingredients extracted from the trichodesma khasianum clarke leaf are carried out for the design of alcohol liver injury animal experiments.

In the design of this study, male mice of C57BL/6J strain are eight weeks old and are pre-bred for two weeks before starting the experiment to allow the animals to adapt to the environment, and observe whether the animals are abnormal daily. Raising at a relative humidity of 60%, 23±1° C., 12-hour light cycle (8:00-20:00), unlimited feed and water for rodents. The establishment and experimental grouping of liver injury and fatigue model animals. The trichodesma khasianum clarke leaf extract powders explore the effect of alcohol liquid diet on fatty liver and liver damage in mice, male mice of the C57BL/6J strain of 8 weeks old, are given special diet for experimental animals (Laboratory Rodent Diet) during the 2 week adaptation period. After one week, the body weight reached 20g, and the group is tested and started to be fed with standard alcoholic liquid model feed (Lieber-DeCarli Regular Control (DYET#710027)) or ethanol feed (Ethanol Diet (DYET#710260)), food and water are not restricted. The test is carried out for six weeks. The test substance is given a fixed daily dose by tube feeding. The dosage conversion refers to the US Food and Drug Administration announcement (US FDA, 2005), based on a 60 kg adult, the recommended daily body weight per kg 12.3 times the intake is the dose for mice. The animal experiment is divided into five groups, and each group has 8 mice, so there are 32 mice, including normal group (NOR group), alcohol group (ALD group), low dose of the trichodesma khasianum clarke leaf extract powder (TL group) and high dose group of the trichodesma khasianum clarke leaf extract powder (TH group), and rosmarinic acid group (RA group). The recommended daily intake of the trichodesma khasianum clarke leaf extract powders are 2 g per day. After the animal experiment dose is converted, the one-time dose is 0.287 g/kg, and the three-time dose is 0.82 g/kg. After fasting for 12 hours, the experimental animals are sacrificed by carbon dioxide asphyxiation, and blood and liver tissues are collected. After the blood is centrifuged, the serum is collected for biochemical analysis. The surface of the tissue is cleaned with saline solution, dried with a paper towel and weighed, then the tissue of the second largest leaf of the liver is cut and placed in a tissue embedding basin soaked in 10% formalin for paraffin embedding and sectioning so as to have tissue staining analysis. Finally, all remained livers are cleaned internally with 0.9% saline and stored in a −80° C. refrigerator for later use. The aforementioned test items of liver function in serum are aspartate aminotransferase (AST), alanine aminotransferase (ALT) and alkaline phosphatase (ALP) which are tested by using biochemical automatic analyzer (Beckman-700, Fullerton, Calif., USA). The test items of a concentration of lipid in blood are total cholesterol (TC) and triglyceride (TG) in serum, wherein a concentration of the total cholesterol is analyzed by using commercially available biochemical reagents (BXC 0261, Fortress), and a concentration of the biochemical reagents (BXC 0271, Fortress) is analyzed by triglyceride. The test items of a concentration of liver lipid are total cholesterol and triglycerides, wherein a concentration of the total cholesterol of the liver lipid is analyzed by using commercially available biochemical reagents (BXC 0261, Fortress), and a concentration of the biochemical reagents (BXC 0271, Fortress) of the liver lipid is analyzed by triglyceride. A production of lipid peroxide malondialdehyde (MDA) is determined by way of thiobarbituric acid (thiobarbituric acid) color method, wherein the more the product of lipid peroxide malondialdehyde is, the higher the absorbance value produces. Therefore, when the sample is capable of reducing the absorbance value of the system, it means that an anti-oxidant effect is formed. The lipid peroxidation analysis is executed based on the method of Tarladgis et al. which using 1,1,3,3-tetramethoxypropane (1,1,3,3-tetramethoxypropane) as a standard product, wherein tetramethoxypropane is diluted to 1 mM with double distilled water, and a concentration of tetramethoxypropane is serially diluted by using one equivalent concentration (1N) sulfuric acid (H2SO4), and 100 μL of which is acquired. The analysis method is the same as that of the sample, for example, taking 50 μL of tissue homogenizer, adding 300 μL of 5% trichloroacetic acid and 100 μL 60 mmol/L thiobarbituric acid. After reacting at 95° C. for 30 minutes, it is cooled in a room temperature and is centrifuged in the room temperature for 20-30 minutes. Thereafter, an absorbance value is

measured, compared with an absorbance value of the standard solution, and a protein concentration is converted to obtain a concentration of lipid peroxide malondialdehyde (Tarladgis et al., 1964).

Analysis Table of Trichodesma Khasianum Clarke Leaf Extracts

| Analyzed Item | Extract Tablets | Analytical method |
|---|---|---|
| Rosmarinic acid | 1.78 | HPLC |
| (mg/per serving) | | |
| Total flavonoids | 34.1 | Chromogenic |
| (mg/per serving) | | |
| Number of bacteria | <10³ CFU | Food and Drug Administration |
| (CFU/g) | (conform) | Authorized inspection method No. |
| | | 1011902832 |
| Escherichia coli | not detected | Ministry of Health and Welfare |
| | (conform) | Authorized inspection method No. |
| | | 1021951163 |
| Escherichia coli | not detected | Food and Drug Administration |
| group | (conform) | Authorized inspection method No. |
| | | 1011902820 |

1.4g of Trichodesma Khasianum Clarke Leaf Extracts powder/per serving

Past studies have shown that fed with alcohol liquid diet in the animal model, the weight gain of the group that consumes alcohol liquid diet will be significantly reduced (Duly et al. 2015).

In the results of this study (Table B1-1), the groups taking alcohol liquid feed (NOR, ALD, TL, TH and RA groups), the average intake of each animal during the test period is 13 mL of alcohol. The results of the weight change (Table B 1-2) showed that the weight of 0 to 6 weeks is significant when the alcohol feed diet and the trichodesma khasianum clarke leaf extract and rosmarinic acid fed at the same time is lower than NOR group (p<0.05). There is no significant difference in body weight compared with the alcohol group (p>0.05), indicating that alcohol intake will reduce body weight, and the main reason is related to total calorie intake.

For example, when alcohol reduces food intake, intake is reduced. The NOR group averages 531 kcal, and the ALD group decreases to 314 kcal, so when a reduced calorie intake occurs, body weight is decreased.

TABLE B1-1
Calorie intake and total alcohol intake of
experimental animals in each group

| | Total calories | Total amount of alcohol |
|---|---|---|
| Groups | (kcal/mouse) | (mL/mouse) |
| NOR | 531.5 | 0.0 |
| ALD | 314.0 | 12.7 |
| TL | 322.0 | 13.2 |
| TH | 316.6 | 13.0 |
| RA | 316.3 | 13.0 |

TABLE B1-2
Changes in body weight of mice with alcohol-induced liver
injury during animal experiments

| | Body weight (g) | | | | | | |
|---|---|---|---|---|---|---|---|
| Groups | Day 0 | Day 7 | Day 14 | Day 21 | Day 28 | Day 35 | Day 42 |
| NOR | 23.4 ± 1.2^{a} | 23.3 ± 1.2^{b} | 24.1 ± 1.2^{c} | 25.3 ± 1.1^{b} | 25.4 ± 0.8^{b} | 26.1 ± 0.9^{b} | 27.1 ± 1.2^{b} |
| ALD | 23.1 ± 0.9^{a} | 21.1 ± 0.9^{a} | 21.3 ± 10.7^{ab} | 23.6 ± 1.1^{ab} | 22.0 ± 1.2^{a} | 22.1 ± 1.1^{a} | 25.4 ± 1.1^{a} |
| TL | 23.3 ± 1.4^{a} | 21.7 ± 1.0^{a} | 22.3 ± 0.8^{b} | 24.1 ± 0.9^{ab} | 22.4 ± 0.8^{a} | 22.3 ± 1.1^{a} | 25.4 ± 1.2^{a} |
| TH | 22.7 ± 1.1^{a} | 21.4 ± 0.5^{a} | 21.4 ± 1.0^{ab} | 23.7 ± 1.2^{a} | 21.7 ± 1.1^{a} | 21.7 ± 1.0^{a} | 24.9 ± 0.9^{a} |
| RA | 22.3 ± 0.7^{a} | 21.7 ± 0.7^{a} | 20.6 ± 0.5^{a} | 23.3 ± 0.8^{a} | 22.3 ± 0.7^{a} | 21.6 ± 0.8^{a} | 24.6 ± 1.0^{a} |

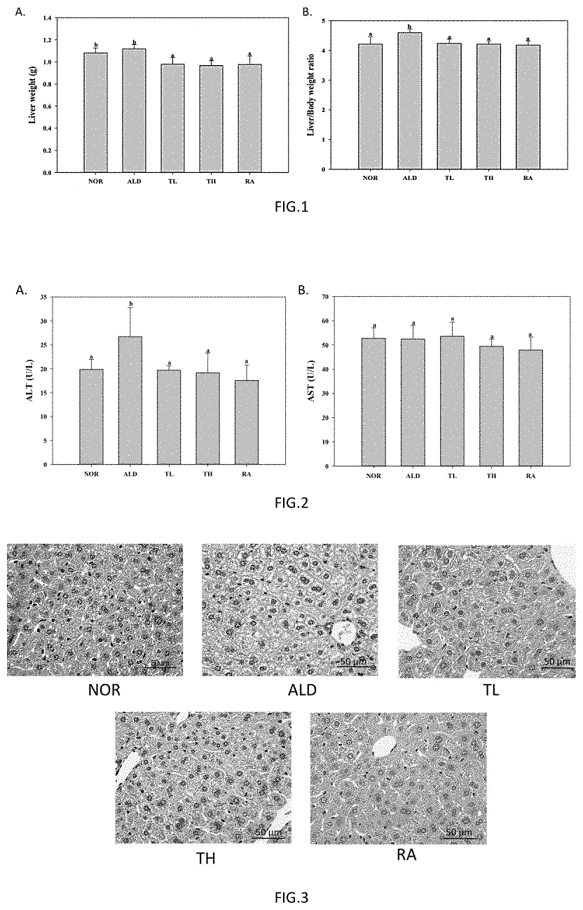

Ingestion of alcohol causes abnormal fatty acid metabolism in the liver, triglycerides accumulate excessively in the cells and aggregate into larger fat droplets to accumulate in the liver, thus promoting hepatomegaly (Orman et al. 2013; Szabo and Mandrekar 2010). Therefore, the degree of liver damage is judged by the relative weight of the liver to the body weight. The results of this study are shown in FIG. 1 that the body weight of the mice in the ALD group receiving alcohol liquid diet is significantly lower than that in the NOR group (p<0.05). Although the liver weight of the alcohol diet in the ALD group is also significantly higher than that of the NOR group (p<0.05), the ratio of liver weight/body weight in the ALD group is significantly higher than that in the NOR group (p<0.05), when feeding the trichodesma khasianum clarke leaf extract. The ratio of liver weight to liver weight/body weight in the RA group is significantly lower than that in the ALD group (p<0.05), it indicates that improving liver enlargement in mice caused by alcohol feed is possible.

The activities of aspartate transaminase and alanine transaminase in the blood are important indicators for examining liver injury (Dufour et al. 2000; Yang et al. 2009). The distribution of alanine transaminase and aspartate transaminase in liver cells is different. Alanine transaminase is mainly distributed in liver cytoplasm, and the increase of alanine transaminase reflects the damage of liver cell membrane. Aspartate transaminase is mainly distributed in liver cytoplasm and liver cell mitochondria. Its increase indicates that liver cells have damaged the organelles of the cell. It can be seen from FIG. 2 that the ALT activity in the serum of the ALD group is significantly higher than that of the NOR group (p<0.05), it means that alcohol induces liver cell damage, but the value of aspartate transaminase has not been significantly increased. The trichodesma khasianum clarke leaf extracts group (TL and TH group) and RA group both significantly reduced the alcohol diet. The induced alanine transaminase activity (p<0.05) shows that it has the potential to improve the alanine transaminase activity induced by alcohol diet, and has the potential to reduce alcohol-induced liver damage.

Aspartate transaminase is mainly distributed in liver cytoplasm and liver cell mitochondria. Its increase indicates that liver cells have damaged the organelles of the cell. It can be seen from FIG. 2 that the ALT activity in the serum of the ALD group is significantly higher than that of the NOR group (p<0.05), it means that alcohol induces liver cell damage, but the value of aspartate transaminase has not been significantly increased. The trichodesma khasianum clarke leaf extract group (TL and TH group) and RA group both significantly reduced the alcohol diet The induced alanine transaminase activity (p<0.05) shows that it has the potential to improve the alanine transaminase activity induced by alcohol diet, and has the potential to reduce alcohol-induced liver damage.

The results of this study are shown in Table B1-3. The results of serum lipid determination found that the serum total cholesterol and triglycerides of the ALD group are significantly higher than those of the NOR group (p<0.05), so it indicates that alcohol induction will make the serum total cholesterol and triglycerides Ester content increased. Furthermore, the trichodesma khasianum clarke leaf extract group (TL group) can only significantly reduce the serum total cholesterol concentration, but there is no significant difference in the reduction of triglyceride content (p>0.05), the trichodesma khasianum clarke leaf extracts. Both group (TH group) and RA group significantly reduce the serum total cholesterol and triglyceride levels raised by alcohol diet (p<0.05). In terms of liver (Table B 1-4), the ALD group significantly increase liver total cholesterol and triglyceride content (p<0.05). Based on the above results, the trichodesma khasianum clarke leaf extract inhibit the lipid content raised by alcohol at low doses, reduce the total serum cholesterol, and in the prevention of fatty liver, the trichodesma khasianum clarke leaf extract reduce the triglyceride content in the live, and reduce the accumulation of lipids in the liver, thus reducing the risk of fatty liver. Rosmarinic acid is one of the main functional components.

TABLE B1-3
Effect of Trichodesma Khasianum Clarke Leaf Extract
on Serum Cholesterol and Triglycerides in Mice with
Alcoholic Liver Injury

| | Serum total | Serum |
|---|---|---|
| | cholesterol | triglyceride |
| Groups | (mg/dL) | (mg/dL) |
| NOR | 87.8 ± 8.58 ^{a} | 85.9 ± 10.9 ^{a} |
| AFLD | 121.5 ± 6.67 ^{b} | 132.4 ± 13.4 ^{c} |
| TL | 89.4 ± 9.89 ^{a} | 111.9 ± 14.1 ^{b} |
| TH | 93.9 ± 13.04 ^{a} | 122.4 ± 16.2 ^{bc} |
| RA | 105.9 ± 9.97 ^{ab} | 107.7 ± 23.5 ^{b} |

TABLE B1-4
Effect of Trichodesma Khasianum Clarke Leaf Extract
on Cholesterol and Triglycerides in the Liver of Mice
with Alcoholic Liver Injury

| | Liver total | Liver |
|---|---|---|
| | cholesterol | triglyceride |
| Groups | (mg/g) | (mg/g) |
| NOR | 2.0 ± 0.20 ^{a} | 5.2 ± 2.44 ^{a} |
| ALD | 8.2 ± 0.52 ^{d} | 13.4 ± 2.30 ^{d} |
| TL | 6.2 ± 0.55 ^{b} | 7.9 ± 2.65 ^{b} |
| TH | 7.1 ± 0.46 ^{c} | 10.4 ± 2.08 ^{c} |
| RA | 6.3 ± 0.56 ^{b} | 8.4 ± 1.68 ^{bc} |

The oxidative stress caused by alcohol will further promote liver lipid peroxidation, and a content of lipid peroxide malondialdehyde (MDA) reflects the degree of lipid peroxidation in the body, and it also shows the severity and measurement of cell oxidative damage. The degree of oxidation in the body (Louvet and Mathurin 2015).

The results of this study are shown in Table B 1-5. After feeding mice with alcohol liquid diet for six weeks, the liver lipid peroxidation malondialdehyde content of the ALD group is significantly increased in the NOR group (p<0.05), it indicates that alcohol causes liver lipid peroxidation. The TH group and the RA group have a tendency to reduce the production of liver lipid peroxides induced by alcohol diet, but there is no significant

difference (p>0.05).

TABLE B1-5
Effect of trichodesma khasianum clarke leaf extract powders on
Liver Lipid Peroxidation in Mice with Alcoholic Liver Injury

| Groups | MDA (mM/g) |
|---|---|
| NOR | 3.97 ± 0.16 ^{a} |
| ALD | 5.97 ± 1.76 ^{b} |
| TL | 5.70 ± 0.38 ^{b} |
| TH | 4.98 ± 0.91 ^{ab} |
| RA | 5.00 ± 0.90 ^{ab} |

C57BL/6J male mice fed with alcohol liquid diet for six weeks are sacrificed and liver sections are stained with hematoxylin ₁₄ and eosin ((H&E stain, hematoxylin and eosin stain)) to assess liver damage.

The results are shown in FIG. 3. The cells in the NOR group are arranged regularly, and the cell structure is intact and compact. On the contrary, the ALD group fed with alcohol liquid diet has many small round fatty vacuoles (microvesicula steatosis), and the cell arrangement is also relatively irregular, but the test substance fed falsely. There are only a few fat vacuoles and macrophage infiltration around the central vein in the trichodesma khasianum clarke leaf extract and RA group, so it shows an improved effect significantly. The slice results of the trichodesma khasianum clarke leaf extract group are closer to the NOR group, and no serious lipid accumulation in the liver is found. It shows that the trichodesma khasianum clarke leaf effectively protects the liver from alcohol damage (2).

Sum up the results of the previous research, and the conclusion is that the trichodesma khasianum clarke leaf extract (TL and TH group) and rosmarinic acid (RA group) significantly reduces alcohol-induced liver enlargement in mice (p<0.05). In improving the liver function index, the trichodesma khasianum clarke leaf extract (TL and TH group) and rosmarinic acid (RA group) significantly reduce the serum liver function index ALT (GPT) activity in alcohol-induced mice (p<0.05). In improving hyperlipidemia caused by alcohol diet, the trichodesma khasianum clarke leaf extract (TL and TH groups) significantly reduce serum cholesterol in alcohol-induced mice (p<0.05). In improving hyperlipidemia caused by alcohol diet, the trichodesma khasianum clarke leaf extract (TL group) and rosmarinic acid (RA group) significantly reduce the serum triglyceride concentration of mice induced by alcohol (p<0.05). In the prevention of fatty liver, pseudophysalis leaf extract (TL and TH group) and rosmarinic acid (RA group) significantly reduce liver cholesterol and triglyceride concentrations in mice (p<0.05). According to liver pathological examination, the trichodesma khasianum clarke leaf extract (TL and TH group) and rosmarinic acid (RA group) improves the effects of alcoholic fatty liver and cell damage in mice. The extraction steps of the trichodesma khasianum clarke leaf include drying, coarse crushing, alcohol extraction, vacuum concentration, freeze drying, fluidized bed granulation, granulation, and tableting; the extract is further added with sea salt, sugar, and mint to have better eating taste (3).

Extracting the trichodesma khasianum clarke leaf and reducing pressure concentration conditions, wherein the trichodesma khasianum clarke leaf is used as a raw material, alcohol extraction is carried out according to different concentrations of alcohol, and the total flavonoids and rosmarinic acid concentration of the extract are determined. After 6 sets of condition tests, it is seen from Table Al-1 that alcohol concentrations higher than 60% and lower than 40% are less effective in extracting total flavonoids and rosmarinic acid, and 60% alcohol is the best extraction efficiency in this study. So the alcohol concentration is chosen to be 50β660% for subsequent large-scale manufacturing processes.

TABLE Al-1
The effect of ethanol concentration on the content
of extracted trichodesma khasianum clarke leaves

| | Alcohol | Total flavonoids | Rosmarinic |
|---|---|---|---|
| Test A | concentration(%) | concentration(mg/g) | acid(mg/g) |
| Condition 1 | 0 | 29.9 ± 3.0 | 0 |
| Condition 2 | 20 | 37.8 ± 5.6 | 0.65 |
| Condition 3 | 40 | 69.1 ± 10.9 | 3.28 |
| Condition 4 | 60 | 82.8 ± 2.9 | 3.9 |
| Condition 5 | 80 | 52.7 ± 3.1 | 0.85 |
| Condition 6 | 95 | 13.4 ± 0.3 | 0.31 |

The above experiment shows that 50β660% alcohol is used to extract the total flavonoids and rosmarinic acid of the trichodesma khasianum clarke leaf. The effect of temperature and time on the concentration of total flavonoids and rosmarinic acid in the extract is further discussed.

From the results of Table Al-2, it is known that the increase of extraction temperature effectively increases the concentration of total flavonoids and rosmarinic acid. From the perspective of the increase per unit time, the extraction time of total flavonoids is one hour, which has a higher extraction efficiency, and then it starts to slow down. Rosmarinic acid has higher extraction efficiency in 1.5 hours. Therefore, the heating temperature is about 60° C. to 75° C. and the time is 1 hour to 1.5 hours during the subsequent large-scale processing, which has a better extraction effect (4).

TABLE A1-2
discusses the influence of temperature and time on the
extract components of trichodesma khasianum clarke leaf

| | Temperature | | Total flavonoids | Rosmarinic |
|---|---|---|---|---|
| Test B | (° C.) | Time | concentration(mg/g) | acid(mg/g) |
| Condition 7 | 45 | 0.5 | 21.5 ± 0.1 | 1.1 ± 0.2 |
| Condition 8 | 45 | 1 | 34.4 ± 1.2 | 1.8 ± 0.1 |
| Condition 9 | 45 | 1.5 | 44.1 ± 1.6 | 2.4 ± 0.2 |
| Condition 10 | 45 | 2 | 50.7 ± 1.7 | 2.8 ± 0.1 |
| Condition 11 | 60 | 0.5 | 36.6 ± 5.1 | 2.3 ± 0.6 |
| Condition 12 | 60 | 1 | 50.5 ± 5.0 | 2.8 ± 0.7 |
| Condition 13 | 60 | 1.5 | 63.1 ± 9.3 | 3.6 ± 0.9 |
| Condition 14 | 60 | 2 | 67.3 ± 13.6 | 4.1 ± 0.7 |
| Condition 15 | 75 | 0.5 | 70.4 ± 4.0 | 3.7 ± 0.4 |
| Condition 16 | 75 | 1 | 91.3 ± 2.8 | 3.9 ± 0.8 |
| Condition 17 | 75 | 1.5 | 103.5 ± 5.2 | 5.1 ± 0.3 |
| Condition 18 | 75 | 2 | 111.5 ± 5.2 | 5.4 ± 0.4 |

According to the above test results, it is known that 60% alcohol, 75° C. stirring extraction, and extraction time of 1 hour - 1.5 hours have a good extraction effect. The concentration of total flavonoids and rosmarinic acid in the trichodesma khasianum clarke leaf can withstand the processing temperature of 75° C.

Therefore, the subsequent concentration conditions can be set at 60° C. to increase the efficiency of vacuum concentration. The extract after vacuum concentration will have a thicker paste and is spray dried directly, and it must be freeze-dried to increase the concentration of active ingredients. After crushing into powder, the fluidized bed granulation is carried out.

While the preferred embodiments of the invention have been set forth for the purpose of disclosure, modifications of the disclosed embodiments of the invention as well as other embodiments thereof may occur to those skilled in the art. The scope of the claims should not be limited by the preferred embodiments set forth in the examples, but should be given the broadest interpretation consistent with the description as a whole.

What is claimed is:

1. A method of producing trichodesma khasianum clarke leaf extracts, the trichodesma khasianum clarke leaf extracts comprising flavonoids and rosmarinic acid, wherein when producing the trichodesma khasianum clarke leaf extracts, a concentration of alcohol is 50% to 60%, an extracting time is 1 hour to 1.5 hours, and an extracting temperature is 60° C. to 75° C., wherein the trichodesma khasianum clarke leaf extracts are configured to regulate a serum liver function index, a serum cholesterol, and a triglyceride in a user's body.

2. The method as claimed in claim 1, wherein before extracting trichodesma khasianum clarke leaves, the trichodesma khasianum clarke leaves are dried and chopped coarsely, then the trichodesma khasianum clarke leaves are extrcated by using alcohol.

3. The method as claimed in claim 1, wherein after extracting the trichodesma khasianum clarke leaves, the trichodesma khasianum clarke leaves are vacuumed, concentrated, frozen, dried, pelletized in a fluid layer, granulated, and tableting pressed to produce the trichodesma khasianum clarke leaf extrcats which are edible.

4. The method as claimed in claim 1, wherein sea salt, sugar, and mint are added into the trichodesma khasianum clarke leaf extracts to enhance taste.
